(19) United States
(12) Patent Application Publication
(10) Pub. No.: US 2011/0200541 A1
(43) Pub. Date: Aug. 18, 2011

(54) RECOMBINANT PREPARATION OF BROMELAIN INHIBITORS AND BROMELAIN INHIBITOR PRECURSOR

(75) Inventors: Rolf Müller, Blieskastel (DE); Nora Luniak, Saarbrucken (DE); Klaus Eschmann, Kleinblittersdorf (DE)

(73) Assignee: Ursapharm Arzneimittel GmbH, Saarbrucken (DE)

(21) Appl. No.: 12/993,244

(22) PCT Filed: Jul. 27, 2009
(86) PCT No.: PCT/EP2009/005426

§ 371 Date: Mar. 31, 2011

(30) Foreign Application Priority Data
Jul. 31, 2008 (EP) 08013773.0

(51) Int. Cl. A61K 38/02; C07K 14/00; C12P 21/02; A61Q 17/04; A61Q 19/08; A61Q 5/02; A61P 35/00; A61P 9/10; A61P 31/04; A61P 29/00; A61P 7/02; A61P 7/10

(52) U.S. Cl. 424/59; 530/324; 530/350; 530/322; 530/395; 514/1.1; 514/19.3; 514/1.9; 514/2.4; 435/69.2

(57) ABSTRACT

The present invention pertains in general to Bromelain and particularly to the active compounds contained in this complex mixture of proteins. The present invention provides recombinant expressed Bromelain inhibitor precursor and Bromelain inhibitors, which are found in Bromelain. It has been found that the recombinant expressed inhibitors have superior effects in terms of treatment of disorders and conditions than Bromelain or its protein fractions from plant extracts.

The present invention pertains in general to Bromelain and particularly to the active compounds contained in this complex mixture of proteins. The present invention provides recombinant expressed Bromelain inhibitor precursor and Bromelain inhibitors, which are found in Bromelain. It has been found that the recombinant expressed inhibitors have superior effects in terms of treatment of disorders and conditions than Bromelain or its protein fractions from plant extracts.

Bromelain is defined biochemically as crude extract from pineapple stem and pharmacologically as a mixture of cysteine proteases. Its multitude of positive effects arises at least in part from the proteolytic and particularly fibrinolytic properties. Anti-tumour effects of Bromelain are also known dependent from effects different from proteolytic activity and still of unknown mechanism. Bromelain is the collective name for the proteolytic enzymes found in the tissues, particularly stem and fruit, of the plants of the Bromeliaceae family. The most common form of Bromelain is a mixture of various moieties derived from the stem of the pineapple plant (Ananas comosus). Stem Bromelain (hereafter called Bromelain) is known to contain at least five proteolytic enzymes but also non-proteolytic enzymes, including an acid phosphatase and a peroxidase; it may also contain amylase and cellulase activity. In addition, various other components are present.

A physical extraction process for Bromelain is e.g. disclosed in CN1186118. The process comprises inter alia pre-treating pineapple plant by freezing, crushing, squeezing to obtain juice, filter to obtain clear liquid, concentrating by ultrafilter film and reverse osmosizing film and freeze vacuum drying to obtain product in the form of sponge. Controlling of temperature, time and pH value is reported to increase enzyme activity and yields.

U.S. Pat. No. 3,658,651 relates Bromelain-containing juice extracted from pineapple plant stems is purified prior to precipitation of the enzyme by passing the juice in ion exchange relation with an anion exchanger in the bicarbonate form, a cation exchanger having weak acid functional groups, and a second anion exchanger in the bicarbonate form.

U.S. Pat. No. 4,286,064 discloses inter alia the preparation of a Bromelain crude extract. The juice from the pineapple stem is first adjusted to a pH of about 3 or 4 with phosphoric acid, and sodium sulfhydride is added to protect against sulfhydryl oxidation. The inert material is precipitated in e.g. acetone and filtrated. The clarified fluid is precipitated with acetone and the precipitate collected by centrifugation and either redissolved in water containing sodium sulfhydride which has been acidified with phosphoric acid and reprecipitated, or dried in a vacuum oven directly. Further purification of the crude extract may be performed by filtration, dialysis or diafiltration for the removal of small molecules and proteins, followed by concentrating the solution obtained prior to lyophilization.

In order to prevent degradation and/or other undesired chemical reactions, such as oxidation reactions, the selection of particular treatment conditions, such as temperature, pH, solvents and buffers, and/or additives, such as stabilizers and antioxidants, is ineluctable.

Apart from the above shortcomings, a further purification of crude extracts may be required. Such purification may be performed for example via HPLC as outlined in US 2002/188107.

It is therefore highly desirable to provide chemically stable as well as pure pharmaceutical active constituents of Bromelain, which may be included in pharmaceutical, dermatological and nutritional compositions and do not exhibit the above shortcomings of the prior art Bromelain containing formulations.

The above problem has been solved by providing heterologously expressed Bromelain inhibitors, wherein said Bromelain inhibitors have been expressed soluble in substantial amounts in a heterologous host.

The present invention is based on the finding that the positive effects assigned to Bromelain may be also accorded to the inhibitors, alone or in combination. The present inhibitors may be obtained with higher purity, particularly avoiding the presence of other proteins or protein fragments, leading to decreased side effects. It has also found that the present Bromelain inhibitors have, even in aqueous solution, higher storage stability over a long period. The present Bromelain inhibitors may be also used for stabilizing any peptide containing pharmaceutical composition, such as antibiotic containing oral compositions.

In course of the present study it has been also found that the precursor protein of the Bromelain inhibitors (Bromelain inhibitor precursor, BIP) represents a natural substrate for the cysteine proteases contained in Bromelain. By heterologous expression of the precursor protein said natural substrate is available in a soluble form of sufficient purity in order to elucidate the activity and specificity of cysteine proteases. As the Bromelain inhibitor precursor protein is degraded inter alia by action of the various cysteine proteases contained in Bromelain to its actual pharmaceutical active constituents, namely several Bromelain inhibitors, said Bromelain inhibitor precursor protein may be also employed as active ingredient in medicaments comprising cysteine proteases originating from e.g. Bromelain. The Bromelain inhibitors emerged from the action of Bromelain inhibit in turn the activity of the cysteine proteases in that the pharmaceutical activity of Bromelain but also any other kind of a cysteine protease containing composition is altered.

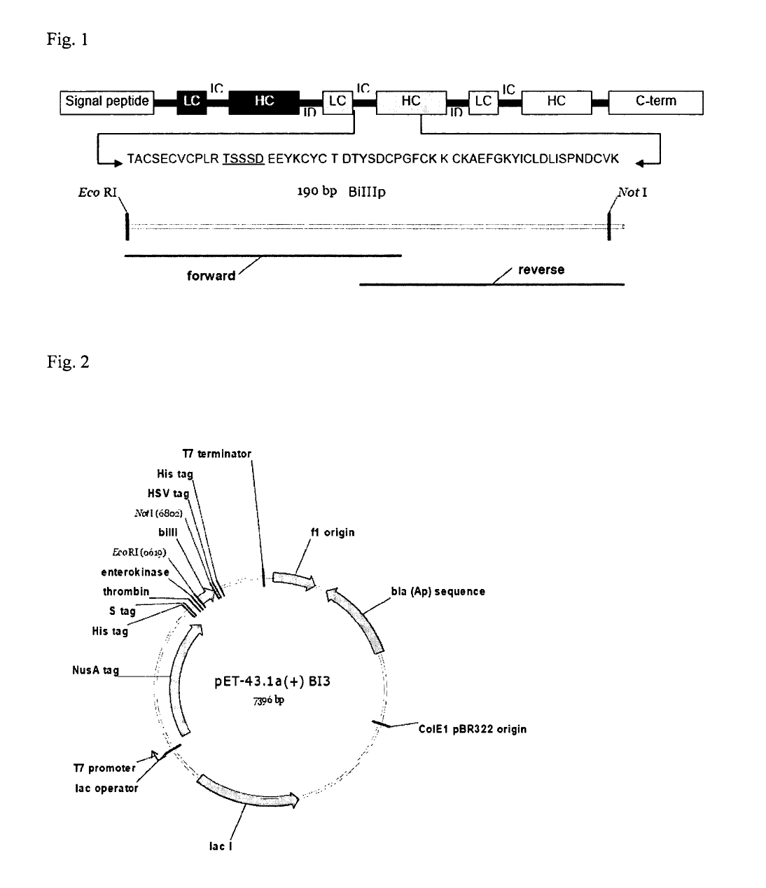

FIG. 1 shows the Bromelain inhibitor III (bi-III) as part of the Bromelain inhibitor precursor (BIP). The shown amino acid sequence corresponds to the light chain with inter chain region and heavy chain.

FIG. 2 shows the expression construct of the Bromelain inhibitor III. The vector (Novagen) employed is designated for E. coli. Selection may be performed via beta-lactamase. Solubility may be significantly enhanced by fusing a NusA-tag.

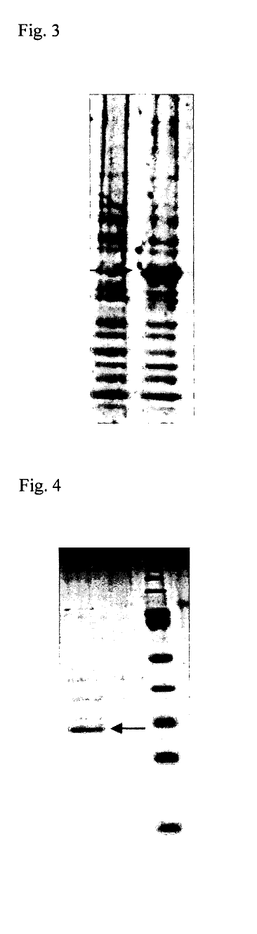

FIG. 3 shows the Bromelain inhibitor in crude extract by E. coli. In particular, a 10% SDS PAGE of the crude extract from pET43.1a/BIP using E. coli Rosetta 2 as expression host. The arrow marks the fusion protein from NusA-tag and Bromelain inhibitor BI-III exhibiting a size of approximately 75 kDa. The left lane refers to the control pET43.1a expressed in E. coli Rosetta 2.

FIG. 4 illustrates expression of the Bromelain inhibitor precursor. In particular, a 10% SDS-PAGE with 100 μl supernatant from Pichia pastoris expression cultures are shown. The left lane illustrates the positive BIP-clone 4, the right negative BIP-clone 5.

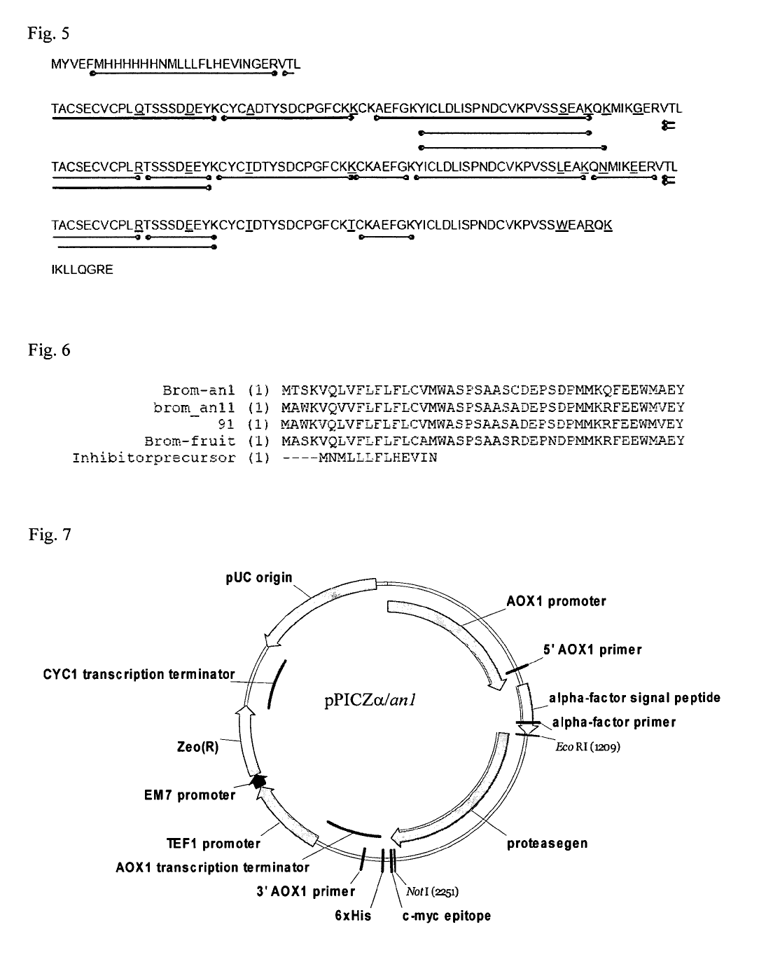

FIG. 5 shows a scheme of the protein sequence of the Bromelain inhibitor precursor with peptides found by peptide mass finger print (underlined).

FIG. 6 shows the ER signal peptide after signal P.

FIG. 7 illustrates the vector pPICZalpha (Invitrogen) comprising the cysteine protease an1. Said construct may be expressed in Pichia pastoris and is may be employed for the quantitative/qualitative assay on Bromelain inhibitor activity.

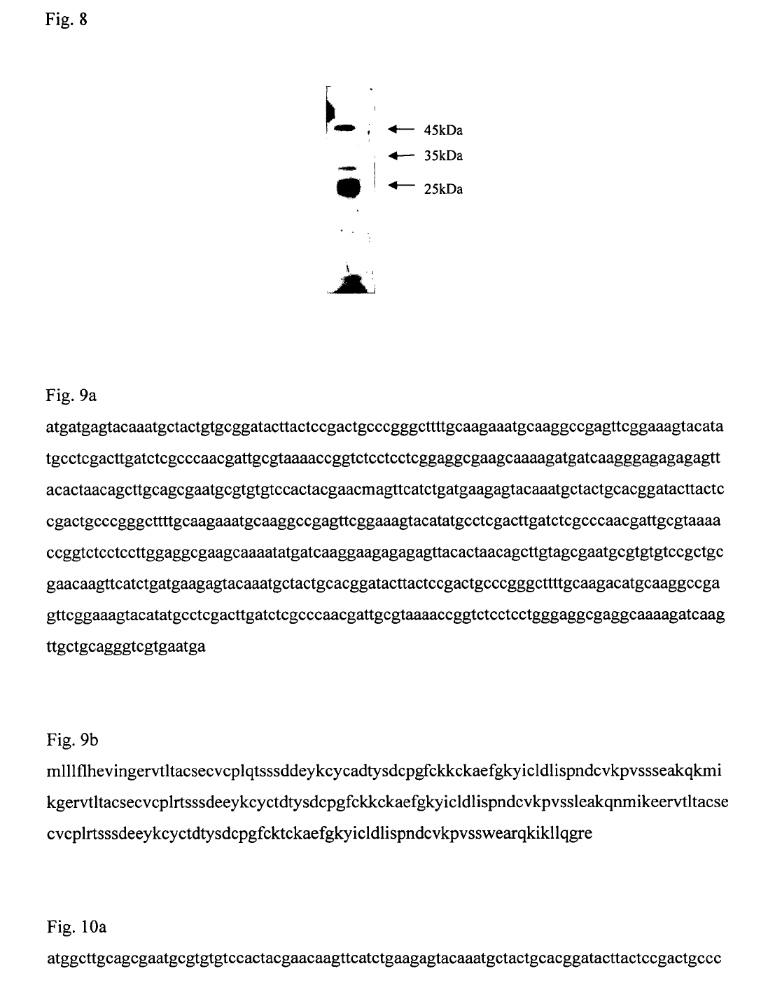

FIG. 8 shows the expression of recombinant Ananain in Pichia pastoris using the construct of FIG. 7. Supernatant of expression culture of Pichia pastoris KM71H with Ananain activated (25 kDa arrow) from expressed Proenzyme (35 kDa arrow) in high yields (45 kDa arrow shows a secreted yeast protein). Recombinant Ananain may be used for activity testing of Bromelain inhibitors.

FIG. 9 shows the DNA (FIG. 9a) and protein (FIG. 9b) sequence of bromelain inhibitor precursor.

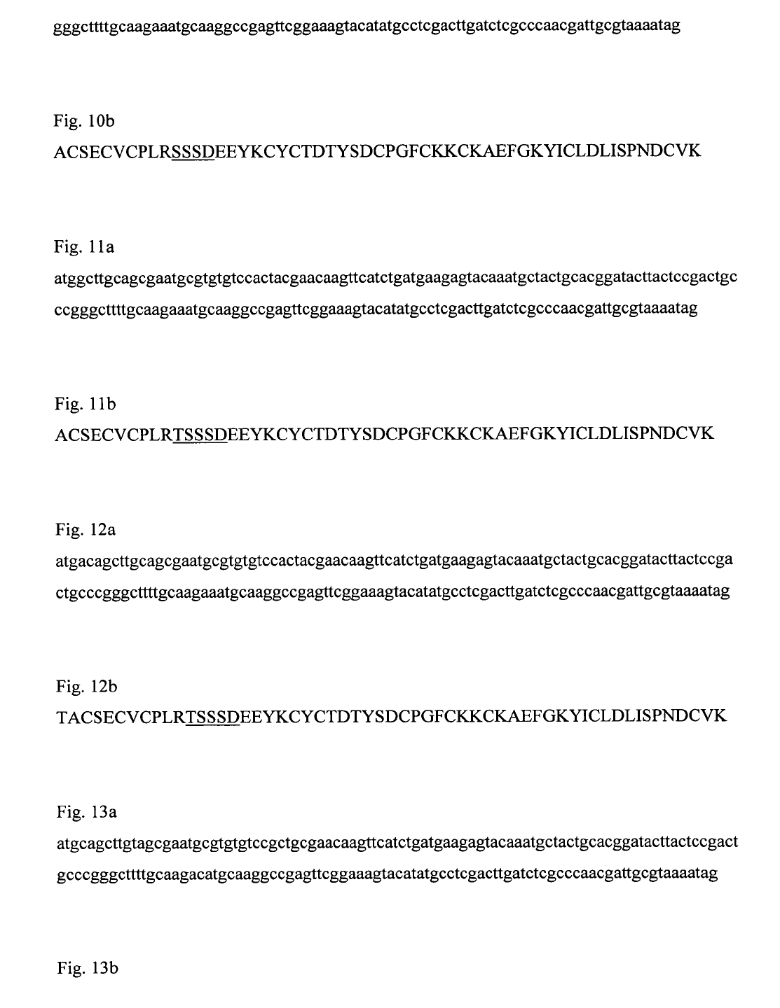

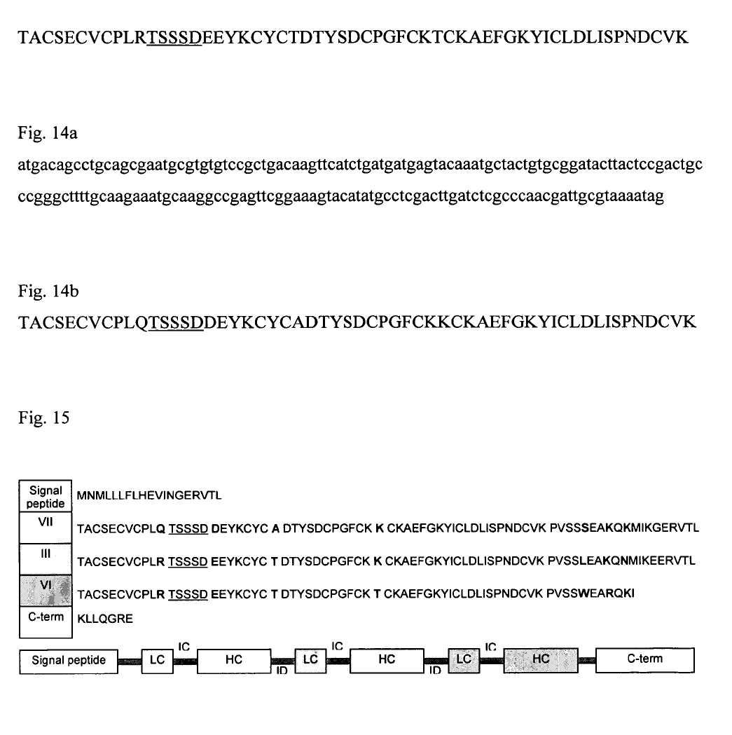

FIG. 10-14 show the DNA sequences (FIG. 10-14a) and protein sequences (FIG. 10-14b) of the single Bromelain inhibitors bi-I, bi-II, bi-III, bi-VI and bi-VII. The DNA sequences include start codon, light Chain, inter chain region, heavy chain and stop codon.

FIG. 15 shows the Bromelain inhibitor precursor Bromein coding for the inhibitor proteins bi-III, bi-VI and bi-VII. The inhibitor proteins are shared by inter domain regions (ID), which are probably cleaved by endopeptidases. An inhibitor consists of light and heavy chain LC and HC, respectively) linked by an inter chain region (IC) representing the “target” of Bromelain and permits auto-regulation of the inhibitor.

According to a first embodiment of the present invention a heterologously expressed Bromelain inhibitor (BI) or Bromelain inhibitor precursor (BIP) is provided, wherein said Bromelain inhibitor (BI) or Bromelain inhibitor precursor (BIP) has been heterologously produced in substantial amounts as soluble protein.

The term heterologously expressed as used herein refers to protein expression in a host organism different from the organism of origin in general and in the present case to expression using as host a genus other than Ananas, i.e. not pineapple plant. Examples for such hosts comprise inter alia Pichia pastoris or E. coli.

Bromelain inhibitor as used herein refers to a single inhibitor contained in any of the fractions of the Bromelain crude extract, which is distinguished from other inhibitors contained in Bromelain by its amino acid sequence. In general, the activity of the inhibitor is preliminary dependent of the active site and its closer surrounding affecting a particular three dimensional structure required for reactivity, whereas more distant stretches of amino acids, such as loops and sheets, may influence the inhibitors substrate affinity and, on the inhibitor's surface, access of the substrate and water solubility.

Bromelain inhibitor precursor refers to the inactive precursor protein of one or more individual Bromelain inhibitors, requiring activation by means of proteases for “releasing” the inhibitors. Activation may be on the one hand bestowed by cysteine proteases and on the other hand by further endopeptidases. A Bromelain inhibitor precursor may be configured as shown in FIG. 15.

The term “substantial amounts” as used herein refers to Bromelain inhibitor precursor or an active Bromelain inhibitor heterologously expressed in an amount of ≧1 mg/l, preferably ≧4 mg/l, ≧8 mg/l, ≧12 mg/l, ≧16 mg/l, ≧20 mg/l, ≧30 mg/l or ≧40 mg/l and more preferably in an amount of ≧50 mg/l.

The term “soluble” as used herein refers to an inhibitor or inhibitor precursor exhibiting essentially the same three dimensional structures as the corresponding native, wild type inhibitor. This ensures that the inhibitor exhibits the desired activity towards cysteine proteases and that the addition of other substances, for e.g. renaturation purposes may be avoided, which ensures on the one hand an improved tolerance upon ingestion and avoids on the other hand undesired modifications of the polypeptide. The inhibitor exhibits further to the surrounding enough hydrophilic amino acids, i.e. undergoing hydrogen bonds with water molecules, in that it exhibits a bioavailability rendering it suitable for use in medical purposes. The term bioavailability describes the rate and extent to which the active ingredient or active moiety is absorbed from a drug product and becomes available at the site of action. The bioavailability of orally ingested drugs is determined by factors, which include the nature of the molecule, its stability, and the formulation administered—and in the patient—such as a reduced intestinal surface area as a result of colic disease or intestinal resection and whether or not the drug is taken with a meal. Factors influencing the bioavailability may include, but are not limited to a poor absorption from the gastrointestinal tract, hepatic first-pass effect and a partial degradation of the drug prior to reaching system circulation. The present expression system permits unexpectedly high yields and an unexpected high solubility of the present inhibitors but also leads to higher amounts of active inhibitor as digestion by proteases is diminished or avoided.

The present Bromelain inhibitors or Bromelain inhibitor precursor may be e.g. obtained by means of RNA isolation from plant material in a first step, e.g. by applying the TRIzol plus RNA Purification System (Invitrogen). DNA as well as proteins may be isolated by means of Trizol (Invitrogen). Greater amounts of RNA from starch rich organs as stem may be isolated using the RNeasy Plant Mini Kit (Qiagen), as starch destroys the gradient needed for separation of RNA from proteins. In a next step a cDNA library may be generated. This may be performed by any method known to the skilled person, such as by employing the SMART™ cDNA Library Construction Kit (Clontech). The cDNA may be ligated in a suitable plasmid or vector followed by transformation in a host organism permitting easy handling, e.g. E. coli. It will be appreciated that this may be performed by any method well known to the skilled person. The inserts may be sequenced by means of standard DNA sequencing techniques. For expression the DNA sequences may be ligated in a suitable expression vector under control of a constitutive or inducible promoter. A suitable expression system is e.g. pPICZalpha (Invitrogen), which may transformed by any suitable technique, such as electroporation, in a suitable host, such as Saccharomyces cerevisiae, or preferably Pichia pastoris. Pichia pastoris as host for heterologous expression exhibits the particular advantage that the glycosylation pattern corresponds essentially to those of higher eukaryotes, as homologues of the α-1,3-Mannosyltransferase are not present in said organism. Other hosts for expression may be Candida albicans or insect cell lines. Individual Bromelain inhibitors may be also obtained by providing Bromelain inhibitor precursor, subjecting to a protease containing composition, such as Bromelain, and isolating the individual inhibitors by means of a method well known to the skilled in the art, e.g. by chromatography.

Transformation and subsequent protein expression may be performed according to the protocols available in the state of the art. The required knowledge of recombinant DNA techniques may be derived from Maniatis et al.; Molecular Cloning: A Laboratory Manual 2nd ed., (1989). Activity of the expressed inhibitor may be easily tested in an assay in which cysteine proteases from e.g. Bromelain are employed. Such proteases are capable to use casein as substrate and cleave/degrade it to casein hydrolysate. This conversion may be qualitatively as well as quantitatively determined. Qualitatively, by including casein in a solid matrix and adding the protease to the matrix' surface. Degradation of casein results in appearance of clear halos forming around the protease. Quantitatively, casein may be provided in solution in a given concentration and adding different amounts of protease. The conversion of casein to the hydrolysate may be followed by means of spectrophotometer working at a wavelength in the visible range of e.g. 600 nm. It will be appreciated that the compound to be tested, i.e. a putative Bromelain inhibitor, may be included in a sample and its effect may be determined by comparison with a sample merely comprising the protease and casein, but not the compound to be tested. Accordingly, other suitable assays for the detection of activity of cysteine proteases or hydrolases in common may be modified and adapted. Examples for other assay may be found in Bornscheuer U. T., Kazlauskas, R. J., Hydrolases in Organic Synthesis, Wiley-VCH, Weinheim (Germany), 1999). The required knowledge of recombinant DNA techniques may be e.g. derived from Maniatis et al.; Molecular Cloning: A Laboratory Manual 2nd ed., (1989). The inhibitory effect of a putative Bromelain inhibitor may be also compared to a known specific cysteine protease inhibitor, such as E64 (Merck).

It has been surprisingly found that expression of the inhibitor precursors and inhibitors in E. coli may be rendered possible by removing the ER-transfer peptide. In addition, the yields of the present Bromelain inhibitors/inhibitor precursor were significantly enlarged by a factor 2, preferably factor 3 or 4, more preferably by a factor 5 or more, in comparison to heterologous expression of a Bromelain inhibitor/inhibitor precursor still bearing ER-transfer peptide. Without wishing to be bound by any theory it is assumed that the ER-transfer peptide confers attachment of the protein to the cell membrane, preventing cell division suggesting a toxic effect of the ER-transfer peptide towards the expression host. This permits inter alia the expression of Bromelain inhibitors or Bromelain inhibitor precursor in E. coli without the need to employ folder proteins/chaperones in order to confer a correct three dimensional structure.

Accordingly, in a first aspect of the present invention heterologously expressed Bromelain inhibitor or Bromelain inhibitor precursor is provided, wherein DNA encoding the ER-transfer peptide of the corresponding Bromelain inhibitor or Bromelain inhibitor precursor has been removed. It will be appreciated that determination of the DNA sequences of ER-transfer peptide may be easily determined according to the knowledge of the skilled person.

According to another aspect of the present invention heterologously expressed Bromelain inhibitor or Bromelain inhibitor precursor is provided. The BI or BIP has a posttranslational modification different from that conferred by the genus Ananas depending on the expression system used. By using an organism different from the genus Ananas, a distinct posttranslational modification (PTM) may be ensured. PTM refers in general to a chemical modification of a protein after its translation, wherein messenger RNA is decoded in order to generate a specific polypeptide or protein. PTMs may be classified according to their actions on the translated protein. They may confer addition of functional groups or other proteins/peptides, result in changing of chemical nature of the amino acids forming the protein and result in structural changes. An example of a posttranslational modification is the glycosylation during which saccharides are added to the protein. Particular a different glycosylation may result in a different water solubility, which may in turn positively affect bioavailability of the protein.

It will be also appreciated by those skilled in the art that the functions of Bromelain inhibitors or Bromelain inhibitor precursor may be achieved by a variety of different amino acid sequences, in that in another embodiment the heterologously expressed BI or BIP exhibits a sequence identity to the respective BI or BIP of at least 90%. Preferably, the sequence identity is at least 95%, 98%, 99% or 99.5% and more preferably at least 99.8%.

Such a BI or BIP exhibiting the above mentioned identity to one of the present BIs or BIP, respectively?, is a polypeptide containing changes in amino acid residues that are not essential for activity, i.e. differ in the amino acid sequence from the original Bromelain inhibitor, yet retain biological function or activity. For example, amino acids may be substituted at “non-essential” amino acid residues. A “non-essential” amino acid residue is a residue that can be altered from the wild-type sequence without altering the biological function or the structural folds, whereas an “essential” amino acid residue is required for biological function. Similar functions are often complied by amino acids with similar structural or chemical properties, for example, replacement of leucine with isoleucine. More rarely, a variant may have “non-conservative” changes, for example, replacement of glycine with tryptophan. The same holds true not only for single amino acids residues, but for entire sequences of amino acids that may be added or omitted without altering the biological function of the protein. Hence, similar minor variations may also include amino acid deletions or insertions, or both. Very often, a short amino acid sequence within a much larger polypeptide is principally responsible for the biological activity or function of a protein.

Hence, the present invention also covers homologues of BIs or BIPs. The homology or sequence similarity or sequence identity of protein sequences may easily be determined according to techniques well known in the art, e.g. by using established software or computer programs, e.g. the BLAST (Basic Local Alignment and Search Tool) program based on the work of Altschul, S. F. et al. (J. Mol. Biol.; 215 (1990) 403-410 and Nucleic Acids Res.; 25 (1997) 3389-3402) offering a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real matches easier to distinguish from random background hits. BLAST uses a heuristic algorithm which seeks local as opposed to global alignments and is therefore able to detect relationships among sequences which share only isolated regions of similarity. Said program is based on modified algorithms of Smith and Waterman (J. Mol. Biol.; 147 (1981) 195-197) and Sellers (Bull. Math. Biol.; 46 (1984) 501-514) to find the best segment of identity or similarity between two sequences. When using a sequence alignment program such as BLAST, to determine the degree of sequence homology, similarity or identity, the default setting may be used, or an appropriate scoring matrix, such as BLOSUM or PAM, may be selected to optimize identity, similarity or homology scores.

It will be appreciated that the present recombinantly produced Bromelain inhibitors or Bromelain inhibitor precursor may be used to elucidate the biological activity and role thereof in the plant. This applies in case of inhibitors especially to interactions with cysteine proteases but also other Bromelain inhibitors, whereas the Bromelain inhibitor precursor forming a natural substrate of the cysteine proteases contained in Bromelain may be used e.g. for performing activity assays, or for in-vitro and in-silico research for other natural substrates or substrates with a similar or improved properties.

In order to account for artificial modifications of the amino acid sequence that may be introduced for a variety of reasons, the present invention also encompasses sequences that are not homologues but that share at least a sequence similarity as defined above or the three-dimensional structure or the function of a Bromelain protein according to the present invention. It will be appreciated that particularly by exchange of single amino acids or by exchange of the glycosylation patterns different properties of the Bromelain inhibitors or Bromelain inhibitor precursor may be obtained. One effect of such an exchange may reside e.g. in an improved bioavailability, e.g. by obtaining a higher solubility in comparison to an inhibitor, which does not bear the particular exchange.

In still another embodiment of the present invention, the heterologously expressed Bromelain inhibitor exhibits any of SEQ ID. No. 1-5 (corresponding to the protein sequences shown in FIG. 10-14b). The heterologously expressed Bromelain inhibitor precursor exhibits SEQ ID. No. 6 (corresponding to the protein sequence shown in FIG. 9a).

The yields of protein exhibiting SEQ ID. No. 1-6 are preferably enlarged by omission of the ER-transfer peptide to the extent as indicated above. The DNA sequences corresponding to the proteins of SEQ ID. No. 1-6 are indicated by SEQ ID. No. 7-12 (SEQ ID. No. 7-11 for the DNA sequences shown in FIG. 10-14a, and SEQ ID. No. 12 for the DNA sequences shown in FIG. 9a).

According to an embodiment of the present invention said posttranslational modification distinguishing the heterologously expressed protein from the wild type protein results in a different glycosylation pattern. It is well known to the skilled person that different organisms used for heterologous expression of a polypeptide may confer a different glycosylation pattern affecting inter alia bioavailability of the protein.

According to still another embodiment the posttranslational modification of the heterologously expressed BIs or BIP is conferred by a host organism selected under the group consisting of yeasts, insect cells, plant cells and E. coli. The selection of such an expression host lies within the knowledge of the skilled person and comprises preliminary adapting of the codon usage of the nucleotide sequence encoding the inhibitor or inhibitor precursor in order to ensure a high expression. Codon usage refers to the phenomenon of preferences of different organisms for one of the several codons, i.e. triplet of nucleotides specifying an amino acid residue in a polypeptide, which encode the same given amino acid. In order to circumvent such preference it may be necessary to e.g. chemically synthesize the DNA encoding the proteins, in which the codon usage is adapted to the chosen expression host. This is within the knowledge of the skilled person. Expression of the BIs or BIP may be preformed according to standard protocols well known to the skilled person. It will be appreciated that conditions for expression, comprising inter alia temperature, medium, kind of vessel, aeration, etc., are within the knowledge of the skilled person and may be easily adapted to the respective requirements.

As a particular suitable expression host, Pichia pastoris has been approved. Such strains have been found not to disturb expression of BIs or BIP as well as respective assays employing a cysteine protease and casein as indicated above. Preferred P. pastoris strains are KM71 or KM71 H, which permit integration in the AOX2-Locus and slow methanol metabolisation. It is assumed that by the slow methanol consumption and corresponding slow production rate of BIs or BIP, correct folding and secretion of the inhibitor is facilitated. Expression in Pichia pastoris using a suitable plasmid, such as pPIC9 or pPICZalpha (both from Invitrogen), ensured high transcription and translation rates. In addition, the Bromelain inhibitor or Bromelain inhibitor precursor maintained solubility and further degradation of the active peptide was reduced or even avoided. The present BIs or BIP exhibited further no significant toxicity to this host organism. Another advantage of using a yeast, such as Pichia pastoris, as expression host resides in the possibility to perform downscaling of growth experiments, i.e. by using microtiter plates with e.g. 24 wells or even 96 wells instead of flasks. This ensures high throughput for testing on properties, such as the ability to inhibit cysteine proteases but also other proteases. The capability of said proteases to degrade a respective substrate, such as a cysteine protease cleaves casein, may be altered by using one of the present Bromelain inhibitors. The activity of said inhibitor is in turn indicative for an underlying medical activity, which corresponds at least in part to that of Bromelain.

According to still another embodiment of the present invention, the heterologously expressed BIs or BIP carry means permitting purification of the inhibitor. Such techniques are well known to the skilled person and may be for example performed by generating and expressing a fusion peptide of the inhibitor or inhibitor precursor with a particular amino acid sequence, which may reversibly bind to the matrix of a column material. The amino acid sequence may be for example a poly-histidine tag sequence fused to the N- or C-terminus of the peptide to be purified. After protein expression the tag binds to the affinity material and is released by means of an imidazole gradient, thereby separating the fusion protein or fusion peptide from other proteins and protein fragments. If required, the tag may be removed. Such techniques are well known to the skilled person. It will be appreciated that any kind of tag may be used in the present invention.

According to a preferred embodiment the inclusion of one or more of the heterologously expressed Bromelain inhibitors or Bromelain inhibitor precursor in a pharmaceutical, dermatological or nutritional composition is envisaged. It will be understood that in case of Bromelain inhibitor precursor, the composition requires also inclusion of proteases digesting Bromelain inhibitor precursor to the active form. Proteases may be supplied e.g. in form of Bromelain or as mixture of cysteine proteases and other endopeptidases. It will be appreciated that the skilled person may easily alter assays in order to determine protease mixtures capable to cleave Bromelain inhibitor precursor in its active form.

According to another embodiment, the compositions of the present invention are formulated in any suitable manner for ingestion. The nutritional composition may be prepared directly before ingestion or alternatively during the manufacturing process. The nutritional composition in the directly usable form is, however, preferred. The active ingredient is contained in acceptable excipients and/or carriers for oral consumption. The expression “nutrionally or pharmaceutical acceptable carrier” refers to a vehicle, for either nutritional or pharmaceutical use, which delivers the active component to its site of action and will not cause significant harm to the human or animal recipient. The actual form of the carrier is, however, not critical.

Another preferred embodiment of the present invention pertains to the use one or more of the present Bromelain inhibitors and/or Bromelain inhibitor precursor for the preparation of a medicament for the prevention and/or treatment a disease associated with an enlarged cysteine protease expression.

The positive effects from Bromelain inhibitors comprise edeme reducing, hemolytic, fibrinolytic, anti-inflammatory, anti-metastatic and tumor inhibitory properties.

Accordingly, another embodiment pertains to the use one or more of the present Bromelain inhibitors and/or Bromelain inhibitor precursor for the preparation of a medicament for the prevention and/or treatment of cancer, atherosclerosis, bacterial infections, inflammations, thromboses and edema. The cancer is preferably selected from prostate, colon, breast and skin cancers.

It will be appreciated that the Bromelain inhibitor precursor requires a proper activation by e.g. Bromelain as indicated above.

According to yet another embodiment the usage of a Bromelain inhibitor as stabilising agent is envisaged in any peptide comprising composition, such as antibiotic containing pharmaceutical compositions intended for oral application.

As the present Bromelain inhibitors exhibit a similar three dimensional structure like Bowman-Birk-Inhibitors, a usage in similar applications is envisaged.

The Bowman-Birk inhibitor was originally found in soybean, wherein said protein comprises 71-amino acids. BBI exhibits a potential chemopreventive activity contains distinct inhibitory sites for trypsin and chymotrypsin. The exact mechanism by which BBI suppresses carcinogenesis is unknown, its antiproliferative activity appears to be linked to the chymotrypsin inhibitory region.

The above underlies the possible implication of the present inhibitors in blood coagulation and inflammation processes. In addition, inhibition of elastases is contemplated. BBI proteins may further increase the stability of medicaments designated for oral administration and are known to act as insecticides (Qi et al., 2005).

The present invention is illustrated by the following examples without limiting it thereto.

### EXAMPLES

Unless stated otherwise, recombinant DNA techniques have been performed according to Maniatis et al.; Molecular Cloning: A Laboratory Manual 2nd ed., (1989). Purified water was obtained by employing a PURELAB ultra (ELGA).

RNA Isolation

RNA but also DNA and proteins were isolated using the Qiazol kit (Invitrogen) following the manufacture's instructions: Mix 1 ml Qiazol with plant material for 50-100 mg fresh weight. To remove polysaccharides, the mixture was centrifuged 10 min, 12.000×g, at room temperature. The supernatant was incubated 5 min at 30° C. Chloroform was added in an amount of 0.2 ml per ml Qiazol. The solution was mixed thoroughly for 15 sec. The mixture was incubated at 30° C. for min. Then the mixture was centrifuged for 15 min., at 4° C., 2.000×g. The upper phase, containing RNA, was removed and mixed with 0.5 ml isopropanol per ml Qiazol. The mixture was centrifuged 10 min, at 4° C., 12.000×g and the supernatant removed. The RNA-Pellet was washed with 1 ml 75% Ethanol (made with DEPC-water) per ml Qiazol, mixed thoroughly and centrifuged 7500×g, 5 min, 4° C. After removal of the supernatant, the pellet was resuspended in RNase free water.

Greater amounts of RNA free from DNA and proteins from pine apple stem were obtained using the RNeasy Plant Mini Kit (Qiagen).

Preparation of a Genomic cDNA Library

For preparation of the genomic cDNA library the SMART IV™ cDNA Library Construction Kit (Clontech) was used. First and second strand synthesis, digestion with proteinase K, digestion with SfiI, cDNA size exclusion fractionation, ligation of the SfiI cut cDNA in SfiI cut, dephosporylated pDNR-LIB vector and transformation of the obtained insert containing vector was performed according to the manufacture's instructions.

Preparation of PCR Products

The PCR reaction for colony PCR comprised a single step of 94° C. for 5 min. and 30 cycles of each 94° C. for 1 min+54° C. for 1 min+72° C. for 1 min, followed by 72° C. for 7 min. and storage at 4° C.

PCR reactions for amplification of sequences for cloning purposes comprised a single step of 98° C. for 1 min. and 25 cycles of each 98° C. for 8 sec.+59° C. for 20 sec.+72° C. for 25 sec., followed by 72° C. for 5 min. and storage at 4° C. The primers employed are listed in table I. BI-for and BI-rev indicate the primers for the precursors the remaining primers are directed to expression of individual Bromelain inhibitors/isoforms.

TABLE 1

| | | | | |
|---|---|---|---|---|
| 82 | BI-for | AATCAAGAATTCATGAACA | Amplif. des Bromelain inhibitor | NL |
| | | TGTTGCTGCTCTTTC | precursor according to Sawano 2002, | |
| | | | mit 83 | |
| 83 | BI-rev | TCACTTATGCGGCCGCACT | Amplif. des Bromelain inhibitor | NL |
| | | CATTCACGACCCTGCA | Precursors according to Sawano 2002, | |
| | | | mit 82 | |
| | Bir2 | TAGATGCGGCCGCTATTTTAC | Bromelain inhibitor, expression | JSF |
| | | GCAATCGTTGGGCGAGATCA | | |
| | | AGTCGAGGCATATGTACTTTC | | |
| | | CGAACTCGGCCTTGCATGTCT | | |
| | | TGCAAAAGCCC | | |
| | BiVIIp-f | TCAGTGAATTCATGACAGCCT | Bromelain inhibitor, expression | JSF |
| | | GCAGCGAATGCGTGTGTCCG | | |
| | | CTGCAAACAAGTTCATCTGAT | | |
| | | GATGAGTACAAATGCTACTG | | |
| | | TGCGGATACTTACTCCGCTCC | | |
| | | GACTGCCCGGGC | | |
| | BiVIp-f | TCAGTGAATTCATGACAGCTT | Bromelain inhibitor, expression | JSF |
| | | GTAGCGAATGCGTGTGTCCG | | |
| | | CTGCGAACAAGTTCATCTGAT | | |
| | | GAAGAGTACAAATGCTACTG | | |
| | | CACGGATACTTACTCCGCTCC | | |
| | | GACTGCCCGGGC | | |
| | BiIII-f | TCAGTGAATTCATGACAGCTT | Bromelain inhibitor, expression | JSF |
| | | GCAGCGAATGCGTGTGTCCA | | |
| | | CTACGAACAAGTTCATCTGAT | | |
| | | GAAGAGTACAAATGCTACTG | | |
| | | CACGGATACTTACTCCGACTG | | |
| | | CCCGGGC | | |
| | BiIIp-f | TCAGTGAATTCATGGCTTGCA | Bromelain inhibitor, expression | JSF |
| | | GCGAATGCGTGTGTCCACTAC | | |
| | | GAACAAGTTCATCTGATGAA | | |
| | | GAGTACAAATGCTACTGCAC | | |
| | | GGATACTTACTCCGCTCCGAC | | |
| | | TGCCCGGGC | | |
| | BiIp-f | TCAGTGAATTCATGGCTTGCA | Bromelain inhibitor, expression | JSF |
| | | GCGAATGCGTGTGTCCACTAC | | |
| | | GAACAAGTTCATCTGATGAG | | |
| | | TACAAATGCTACTGCACGGA | | |
| | | TACTTACTCCGCTCCGACTGC | | |
| | | CCGGGC | | |
| | BIr1 | ATGTAGCGGCCGCTATTTTAC | Bromelain inhibitor, expression | JSF |
| | | GCAATCGTTGGGCGAGATCA | | |
| | | AGTCGAGGCATATGTACTTTC | | |
| | | CGAACTCGGCCTTGCATTTCT | | |
| | | TGCAAAAGCCCGGGCAGTCG | | |
| | | GAG | | |

PCR reactions for assembly of isoform sequences for cloning purposes comprised a single step of 94° C. for 5 min. and 25 cycles of each 95° C. for 8 sec.+59° C. for 15 sec.+60° C. for 25 sec., followed by 72° C. for 5 min. and storage at 4° C.

PCR Reactions

The PCR reaction was performed according to manufacturer's instructions in HF buffer for high fidelity with Phusion™ Polymerase.

Phusion-Polymerase; NEB

| | |
|---|---|
| 10 | μl 10 × buffer HF (NEB) |
| 0, 5 | μl primer A (100 pmol/μl) |
| 0, 5 | μl primer B (100 pmol/μl) |
| 0, 5 | μl dNTP-Mix (10 mM, Fermentas) |
| x | μl ddH₂O |
| 0, 2 | μl Phusion (2, 0 U/μl) (NEB) |
| x | ng DNA |
| 50 | μl Σ sterile water |

As template pDNR-LIB containing cDNA fragments was used. Amplified sequenced were subcloned pCRII by TOPO TA Cloning® Kit (Invitrogen), as instructed by manufacturer.

Colony PCR was performed using taq-Polymerase (Fermentas). Therefore all components were pipetted and cell material was added to the reaction.

Colony PCR:

| | |
|---|---|
| 5 | μl 10× buffer |
| 5 | μl 25 mM MgCl₂ |
| 1 | μl 25 mM dNTPs |
| 1 | μl 5′ AOX1 primer (10 pmol/μl) |
| 1 | μl 3′ AOX1 primer (10 pmol/μl) |
| 27 | μl sterile water |
| 5 | μl cell material |
| 45 | μl total volume |

Isoform assembly:
5 μl 10× buffer
5 μl 25 mM MgCl₂
1 μl 25 mM dNTPs
1 μl 5′ primer (10 pmol/μl)
1 μl 3′ primer (10 pmol/μl)
37 μl sterile water
50 μl total volume

The PCR reactions, which were all conducted with sterile PCR reaction vessels (Eppendorf, Hamburg, Germany) in a Mastercycler Gradient (Eppendorf, Germany), were separated by means of a conventional SDS agarose gel, excised and purified by means of the NucleoSpin® Plasmid-DNA Kit (Macherey & Nagel, Düren, Germany) or Microspin Columns (Amersham Pharmacia) for sequencing. The products were cloned in pTOPO-PCRII (Invitrogen). Gels were performed by means of a Mighty Small SE250/SE260 (Hoefer) employing SUB-CELL® GT or MINI-SUB-CELL® GT (both Biorad). As centrifuges the Avanti JE cooling centrifuge (Beckmann Coulter), Biofuge pico or Biofuge fresco (both Heraeus) were used.

Sequencing

DNA Sequencing has been performed according to manufacture's instruction with a capillary sequencer (MWG).

Cloning/Growth Conditions

The sequence of the Bromelain inhibitor BIP (Bromelain inhibitor III) without ER-transfer peptide has been cloned pPET43.1a (Novagen) and cloned in E. coli Rosetta 2 according to the manufactures instructions (cf. FIG. 3). Purification of the inhibitor via affinity chromatography employing Ni-sepharose yielded approximately 40 mg/l of purified inhibitor.

The sequences of the Bromelain cysteine protease ani (Acnr¹: CAA05487) as well as Bromelain inhibitor BIP have been cloned in frame in pPIC9 or pPICZA (both Invitrogen) using EcoRI and NotI restriction sites. Cloning has been performed with a Gene pulser/Pulse Controller (both Biorad) according to the manufacture's instructions.

As control pPICZ/gfp was used, which was kindly obtained by Glieder, TU Graz). Said plasmid comprises gfp (Shimomura O. et al., J. Cell. Comp. Physiol., 59 (1962), 223-239; Shimomura O., J. Microsc., 217 (2005), 1-15), employing the same restriction sites and the same conditions for growth and induction.

Assay on Cystein Protease Inhibition

Bromelain inhibitor was purified from E. coli culture and pre-incubated with Bromelain or trypsin for 2 h. The mixture was treated 20 min, 80° C. to remove proteolytic activity in case of Bromelain pre-incubation or by serine protease inhibitor in case of trypsin. Supernatant from Pichia pastoris containing heterologously expressed, active cysteine proteases from Ananas comosus and casein (0.5%) were be added. OD600 was determined after 1 hour incubation at room temperature using a standard microtiter plate reader. The results of the different samples were compared. Other proteases were used as substrates for inhibitory activity like Bowman Birk inhibitor, trypsin, chymotrypsin, elastase, Falcipain and Bromelain BP. Samples were compared to inactivated protease extract, and appropriate synthetic inhibitor (e.g. E64 in case of cysteinproteases).

P. pastoris comprising pPICZA with alpha factor, propeptide and Ananain (An1) but without C-terminal cysteine protease sequence yielded 12 mg/l active AN 1 (casein as substrate) in 1 l of BMM medium (pH 6) grown for up to 48 h in shaking flasks equipped with three or four baffles at 230 rpm and 28° C., employing a Unitron HT shaking incubator (Infors). The above mentioned tests were also conducted at different temperatures (1 hour incubation at 30, 40, 50, 60, 70 and 80° C., respectively) exhibiting a residual activity of the Bromelain inhibitor at 70° C. of 82% ±8% in comparison to the activity at room temperature (between 22 and 24° C.) (results not shown) underlying the similarity to BBI proteins and a respective use of BIP.

Maldi-Tof Analysis of Bromelain Proteins

Samples from the supernatants were taken after 3 d of cultivation and purified with Zip-Tips (Millipore) before subjecting to Maldi-Tof Analysis employing a 4800 TOF/TOF mass analyser (Applied Biosystems).

It could be derived that the secretion signal was cleaved off in a correct manner. The proteins expressed in P. pastoris exhibited glycosylation as verified with the Glykomod tool (www.ExPASy.ch; results not shown).

1. A heterologously expressed Bromelain inhibitor or Bromelain inhibitor precursor, wherein said Bromelain inhibitor or Bromelain inhibitor precursor is heterologously expressed in a substantial amount in a soluble form.

2. The heterologously expressed Bromelain inhibitor or Bromelain inhibitor precursor according to claim 1, wherein a DNA sequence encoding the an ER-transfer peptide of the Bromelain inhibitor has been removed.

3. The heterologously expressed Bromelain inhibitor or Bromelain inhibitor precursor according to claim 1, wherein said Bromelain inhibitor has a posttranslational modification different from that conferred by the genus Ananas.

4. The heterologously expressed Bromelain inhibitor or Bromelain inhibitor precursor claim 1, wherein the heterologously expressed Bromelain inhibitor or Bromelain inhibitor precursor exhibits a sequence identity to the a respective Bromelain inhibitor or Bromelain inhibitor precursor of at least 90%.

5. The heterologously expressed Bromelain inhibitor or Bromelain inhibitor precursor according to claim 1, wherein the Bromelain inhibitor comprises a sequence of any of SEQ ID. No. 1-5 or the Bromelain inhibitor precursor comprises a sequence of SEQ ID. No 6.

6. The heterologously expressed Bromelain inhibitor or Bromelain inhibitor precursor according to claim 3, wherein said posttranslational modification results in a different glycosylation pattern.

7. The heterologously expressed Bromelain inhibitor or Bromelain inhibitor precursor according to claim 6, wherein the posttranslational modification is conferred by a host organism selected under from the group consisting of yeasts, insect cells, plant cells and E. coli.

8. The heterologously expressed Bromelain inhibitor or Bromelain inhibitor precursor according to claim 1, wherein the Bromelain inhibitor or Bromelain inhibitor precursor carries a component permitting purification.

9. A pharmaceutical, dermatological or nutritional composition comprising heterologously expressed Bromelain inhibitor or Bromelain inhibitor precursor according to claim 1.

10. The pharmaceutical, dermatological or nutritional composition according to claim 9, wherein the heterologously expressed Bromelain inhibitor or Bromelain inhibitor precursor is contained in an amount in a range of from 0.1 to 15 wt.-%, based on the a total weight of the composition.

11. The pharmaceutical, dermatological or nutritional composition according to claim 9,
wherein the nutritional composition is in a form selected from the group consisting of chocolate, milk, yoghurt, curd, cheese, fermented milks, milk based fermented products, ice-creams, fermented cereal based products, milk based powders, infant formulae, and pet food; and/or
wherein the dermatological composition is in a form selected from the group consisting of lotions, shampoos, creams, sunscreens, after-sun creams, anti-aging creams, and ointments; and/or
wherein the pharmaceutical composition is in a form selected from the group consisting of tablets, liquid suspensions, dried oral supplement, wet oral supplement, dry tube-feeding, and wet tube-feeding.

12. The pharmaceutical, dermatological or nutritional composition according to claim 9, wherein said pharmaceutical composition additionally comprises an excipient selected from the group consisting of diluents, binding agents, disintegrants, lubricants, sweeteners, glidants, flavourings and colouring agents.

13. (canceled)

14. (canceled)

15. A method for treatment and/or prophylaxis of a disease associated with an enlarged cysteine protease expression, the method comprising:
administering a pharmaceutical composition comprising the heterologously expressed Bromelain inhibitor or Bromelain inhibitor precursor according to claim 1 to a subject in need of the treatment and/or the prophylaxis.

16. A method for treatment and/or prophylaxis of cancer, atherosclerosis, bacterial infections, inflammations, thromboses and edema, the method comprising:
administering a pharmaceutical composition comprising the heterologously expressed Bromelain inhibitor or Bromelain inhibitor precursor according to claim 1 to a subject in need of the treatment and/or the prophylaxis.

17. A method for producing a heterologously expressed Bromelain inhibitor or Bromelain inhibitor precursor according to claim 1 comprising:
heterologously expressing a polynucleotide encoding said heterologously expressed Bromelain inhibitor or Bromelain inhibitor precursor in a host.

18. The method according to claim 17, wherein a DNA sequence encoding an ER-transfer peptide of the Bromelain inhibitor has been removed.

19. The method according to claim 17, wherein said Bromelain inhibitor has a posttranslational modification different from that conferred by the genus Ananas.

20. The method according to claim 17, wherein the heterologously expressed Bromelain inhibitor or Bromelain inhibitor precursor exhibits a sequence identity to a respective Bromelain inhibitor or Bromelain inhibitor precursor of at least 90%.

21. The method according to claim 17, wherein the polynucleotide sequence encoding said heterologously expressed Bromelain inhibitor or Bromelain inhibitor precursor comprises a fragment of a sequence selected from the group consisting of SEQ ID NOs: 7-12.

22. The method according to claim 19, wherein said posttranslational modification results in a different glycosylation pattern.

23. The method according to claim 22, wherein the posttranslational modification is conferred by a host organism selected from the group consisting of yeasts, insect cells, plant cells and E. coli.


SEQUENCE LISTING (33 sequences)

SEQ ID NO: 1 - protein, 55 aa, Ananas comosus
ACSECVCPLRSSSDEEYKCYCTDTYSDCPGFCKKCKAEFGKYICLDLISPNDCVK

SEQ ID NO: 2 - protein, 56 aa, Ananas comosus
ACSECVCPLRTSSSDEEYKCYCTDTYSDCPGFCKKCKAEFGKYICLDLISPNDCVK

SEQ ID NO: 3 - protein, 57 aa, Ananas comosus
TACSECVCPLRTSSSDEEYKCYCTDTYSDCPGFCKKCKAEFGKYICLDLISPNDCVK

SEQ ID NO: 4 - protein, 57 aa, Ananas comosus
TACSECVCPLRTSSSDEEYKCYCTDTYSDCPGFCKTCKAEFGKYICLDLISPNDCVK

SEQ ID NO: 5 - protein, 57 aa, Ananas comosus
TACSECVCPLQTSSSDDEYKCYCADTYSDCPGFCKKCKAEFGKYICLDLISPNDCVK

SEQ ID NO: 6 - protein, 244 aa, Ananas comosus
MLLLFLHEVINGERVTLTACSECVCPLQTSSSDDEYKCYCADTYSDCPGFCKKCKAEFGKYICLDLISPNDCVKPVSSSEAKQKMIKGERVTLTACSECVCPLRTSSSDEEYKCYCTDTYSDCPGFCKKCKAEFGKYICLDLISPNDCVKPVSSLEAKQNMIKEERVTLTACSECVCPLRTSSSDEEYKCYCTDTYSDCPGFCKTCKAEFGKYICLDLISPNDCVKPVSSWEARQKIKLLQGRE

SEQ ID NO: 7 - DNA, 171 bp, Ananas comosus
atggcttgcagcgaatgcgtgtgtccactacgaacaagttcatctgaagagtacaaatgctactgcacggatacttactccgactgcccgggcttttgcaagaaatgcaaggccgagttcggaaagtacatatgcctcgacttgatctcgcccaacgattgcgtaaaatag

SEQ ID NO: 8 - DNA, 174 bp, Ananas comosus
atggcttgcagcgaatgcgtgtgtccactacgaacaagttcatctgatgaagagtacaaatgctactgcacggatacttactccgactgcccgggcttttgcaagaaatgcaaggccgagttcggaaagtacatatgcctcgacttgatctcgcccaacgattgcgtaaaatag

SEQ ID NO: 9 - DNA, 177 bp, Ananas comosus
atgacagcttgcagcgaatgcgtgtgtccactacgaacaagttcatctgatgaagagtacaaatgctactgcacggatacttactccgactgcccgggcttttgcaagaaatgcaaggccgagttcggaaagtacatatgcctcgacttgatctcgcccaacgattgcgtaaaatag

SEQ ID NO: 10 - DNA, 176 bp, Ananas comosus
atgcagcttgtagcgaatgcgtgtgtccgctgcgaacaagttcatctgatgaagagtacaaatgctactgcacggatacttactccgactgcccgggcttttgcaagacatgcaaggccgagttcggaaagtacatatgcctcgacttgatctcgcccaacgattgcgtaaaatag

SEQ ID NO: 11 - DNA, 174 bp, Ananas comosus
atgacagcctgcagcgaatgcgtgtgtccgctgacaagttcatctgatgatgagtacaaatgctactgtgcggatacttactccgactgcccgggcttttgcaagaaatgcaaggccgagttcggaaagtacatatgcctcgacttgatctcgcccaacgattgcgtaaaatag

SEQ ID NO: 12 - DNA, 638 bp, Ananas comosus
atgatgagtacaaatgctactgtgcggatacttactccgactgcccgggcttttgcaagaaatgcaaggccgagttcggaaagtacatatgcctcgacttgatctcgcccaacgattgcgtaaaaccggtctcctcctcggaggcgaagcaaaagatgatcaagggagagagagttacactaacagcttgcagcgaatgcgtgtgtccactacgaacmagttcatctgatgaagagtacaaatgctactgcacggatacttactccgactgcccgggcttttgcaagaaatgcaaggccgagttcggaaagtacatatgcctcgacttgatctcgcccaacgattgcgtaaaaccggtctcctccttggaggcgaagcaaaatatgatcaaggaagagagagttacactaacagcttgtagcgaatgcgtgtgtccgctgcgaacaagttcatctgatgaagagtacaaatgctactgcacggatacttactccgactgcccgggcttttgcaagacatgcaaggccgagttcggaaagtacatatgcctcgacttgatctcgcccaacgattgcgtaaaaccggtctcctcctgggaggcgaggcaaaagatcaagttgctgcagggtcgtgaatga

SEQ ID NO: 13 - DNA, 34 bp, Artificial sequence
  BI-forward primer
aatcaagaattcatgaacatgttgctgctctttc

SEQ ID NO: 14 - DNA, 35 bp, Artificial sequence
  BI-reverse primer
tcacttatgcggccgcactcattcacgaccctgca

SEQ ID NO: 15 - DNA, 94 bp, Artificial sequence
  Bir2 primer
tagatgcggccgctattttacgcaatcgttgggcgagatcaagtcgaggcatatgtactttccgaactcggccttgcatgtcttgcaaaagccc

SEQ ID NO: 16 - DNA, 115 bp, Artificial sequence
  BiVIIp-f primer
tcagtgaattcatgacagcctgcagcgaatgcgtgtgtccgctgcaaacaagttcatctgatgatgagtacaaatgctactgtgcggatacttactccgctccgactgcccgggc

SEQ ID NO: 17 - DNA, 115 bp, Artificial sequence
  BiVIp-f primer
tcagtgaattcatgacagcttgtagcgaatgcgtgtgtccgctgcgaacaagttcatctgatgaagagtacaaatgctactgcacggatacttactccgctccgactgcccgggc

SEQ ID NO: 18 - DNA, 110 bp, Artificial sequence
  BiIII-f primer
tcagtgaattcatgacagcttgcagcgaatgcgtgtgtccactacgaacaagttcatctgatgaagagtacaaatgctactgcacggatacttactccgactgcccgggc

SEQ ID NO: 19 - DNA, 112 bp, Artificial sequence
  BiIIp-f primer
tcagtgaattcatggcttgcagcgaatgcgtgtgtccactacgaacaagttcatctgatgaagagtacaaatgctactgcacggatacttactccgctccgactgcccgggc

SEQ ID NO: 20 - DNA, 109 bp, Artificial sequence
  BiIp-f primer
tcagtgaattcatggcttgcagcgaatgcgtgtgtccactacgaacaagttcatctgatgagtacaaatgctactgcacggatacttactccgctccgactgcccgggc

SEQ ID NO: 21 - DNA, 106 bp, Artificial sequence
  BIr1 primer
atgtagcggccgctattttacgcaatcgttgggcgagatcaagtcgaggcatatgtactttccgaactcggccttgcatttcttgcaaaagcccgggcagtcggag

SEQ ID NO: 22 - protein, 57 aa, Ananas comosus; identical to SEQ ID NO: 3

SEQ ID NO: 23 - protein, 257 aa, Ananas comosus
MYVEFMHHHHHHNMLLLFLHEVINGERVTLTACSECVCPLQTSSSDDEYKCYCADTYSDCPGFCKKCKAEFGKYICLDLISPNDCVKPVSSSEAKQKMIKGERVTLTACSECVCPLRTSSSDEEYKCYCTDTYSDCPGFCKKCKAEFGKYICLDLISPNDCVKPVSSLEAKQNMIKEERVTLTACSECVCPLRTSSSDEEYKCYCTDTYSDCPGFCKTCKAEFGKYICLDLISPNDCVKPVSSWEARQKIKLLQGRE

SEQ ID NO: 24 - protein, 44 aa, Ananas comosus
MTSKVQLVFLFLFLCVMWASPSAASCDEPSDPMMKQFEEWMAEY

SEQ ID NO: 25 - protein, 44 aa, Ananas comosus
MAWKVQVVFLFLFLCVMWASPSAASADEPSDPMMKRFEEWMVEY

SEQ ID NO: 26 - protein, 44 aa, Ananas comosus
MAWKVQLVFLFLFLCVMWASPSAASADEPSDPMMKRFEEWMVEY

SEQ ID NO: 27 - protein, 44 aa, Ananas comosus
MASKVQLVFLFLFLCAMWASPSAASRDEPNDPMMKRFEEWMAEY

SEQ ID NO: 28 - protein, 13 aa, Ananas comosus
MNMLLLFLHEVIN

SEQ ID NO: 29 - protein, 19 aa, Ananas comosus
MNMLLLFLHEVINGERVTL

SEQ ID NO: 30 - protein, 76 aa, Ananas comosus
TACSECVCPLQTSSSDDEYKCYCADTYSDCPGFCKKCKAEFGKYICLDLISPNDCVKPVSSSEAKQKMIKGERVTL

SEQ ID NO: 31 - protein, 76 aa, Ananas comosus
TACSECVCPLRTSSSDEEYKCYCTDTYSDCPGFCKKCKAEFGKYICLDLISPNDCVKPVSSLEAKQNMIKEERVTL

SEQ ID NO: 32 - protein, 68 aa, Ananas comosus
TACSECVCPLRTSSSDEEYKCYCTDTYSDCPGFCKTCKAEFGKYICLDLISPNDCVKPVSSWEARQKI

SEQ ID NO: 33 - protein, 7 aa, Ananas comosus
KLLQGRE